(19) United States
(12) Patent Application Publication
(10) Pub. No.: US 2022/0072550 A1
(43) Pub. Date: Mar. 10, 2022

(54) CELL MARKING SYSTEMS

(71) Applicant: Hewlett-Packard Development Company, L.P., Spring, TX (US)

(72) Inventors: Alexander Govyadinov, Corvallis, OR (US); Viktor Shkolnikov, Palo Alto, CA (US)

(73) Assignee: Hewlett-Packard Development Company, L.P., Spring, TX (US)

(21) Appl. No.: 17/417,344

(22) PCT Filed: Feb. 1, 2019
(86) PCT No.: PCT/US2019/016353

§ 371 Date: Jun. 22, 2021

(51) Int. Cl. B01L 3/00

(52) U.S. Cl. CPC B01L 3/502761; B01L 2200/10; B01L 2200/0652

(57) ABSTRACT

In one example in accordance with the present disclosure, a cell marking system is described. The cell marking system includes a microfluidic channel to serially feed individual cells from a volume of cells into at least one marking chamber. The at least one marking chambers hold an individual cell to be marked. The cell marking system also includes a marker application device per marking chamber to selectively apply a marker to the individual cell disposed within a respective marking chamber.

### BACKGROUND

In analytic chemistry, scientists use instruments to separate, identify, and quantify matter. Cell lysis is a process of rupturing the cell membrane to extract intracellular components for purposes such as purifying the components, retrieving deoxyribonucleic acid (DNA), ribonucleic acid (RNA), proteins, polypeptides, metabolites, or other small molecules contained therein, and analyzing the components for genetic and/or disease characteristics. Cell lysis bursts a cell membrane and frees the inner components. The fluid resulting from the bursting of the cell is referred to as lysate.

### BRIEF DESCRIPTION OF THE DRAWINGS

The accompanying drawings illustrate various examples of the principles described herein and are part of the specification. The illustrated examples are given merely for illustration, and do not limit the scope of the claims.

FIG. 1 is a block diagram of a cell marking system, according to an example of the principles described herein.

FIG. 2 is a flow chart of a method of cell marking, according to an example of the principles described herein.

FIG. 3 is a diagram of a cell marking system, according to an example of the principles described herein.

FIG. 4 is a block diagram of a cell analysis system, according to an example of the principles described herein.

FIG. 5 is a diagram of a cell analysis device, according to an example of the principles described herein.

FIG. 6 is a diagram of a cell analysis device, according to another example of the principles described herein.

FIG. 7 is a diagram of a cell analysis device, according to another example of the principles described herein.

FIG. 8 is a diagram of a cell analysis device, according to another example of the principles described herein.

FIG. 9 is a diagram of a cell analysis device, according to another example of the principles described herein.

FIG. 10 is a diagram of a cell analysis device, according to another example of the principles described herein.

FIG. 11 is a diagram of a cell analysis device, according to another example of the principles described herein.

FIG. 12 is a diagram of a cell analysis device, according to another example of the principles described herein.

FIG. 13 is a diagram of a cell analysis device, according to another example of the principles described herein.

FIG. 14 is a flow chart of a method of cell marking, according to an example of the principles described herein.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION

Cellular analytics is a field of chemistry that uses instruments to separate, identify, and quantify matter. A wealth of information can be collected from a cellular sample.

For example, the mechanical properties of the cell membrane and even more specifically information relating to the mechanical breakdown of the cell membrane can provide insight to the characteristics and state of a cellular sample. For example, in some cases the physical characteristics of a particular cell can be used to classify and/or differentiate the particular cell from other cells. In another example, changes to the physical characteristics of a cell can be used to determine a state of the cell. For example, parasitic invasion of a cell—such as occurs in cells affected by malaria—can alter the membrane of the cell. Gross changes to tissue, such as when cancer is present in a cell, can also alter the physical properties of the cell membrane. In other words, cell membrane strength indicates cell membrane composition and cell composition. Accordingly, a cell analysis system that can measure cell membrane strength provides to an individual, information regarding the cell membrane composition from which characteristics of the cell can be determined.

The intracellular components of the cell also provide valuable information about a cell. Cell lysis is a process of extracting intracellular components from a cell. During lysis, the intracellular components are extracted for purposes such as purifying the components, retrieving DNA and RNA proteins, polypeptides, metabolites, and small molecules or other components therein, and analyzing the components for genetic and/or disease characteristics. Cell lysis ruptures a cell membrane and frees the inner components. The fluid containing the inner components is referred to as lysate. The contents of the cell can then be analyzed by a downstream system.

The study and analysis of the lysate of a cell provides information used to characterize and analyze a cell. For example, cytoplasmic fluid within the cell may provide a picture of the current mechanisms occurring within the cell. Examples of such mechanisms include ribonucleic acid (RNA) translation into proteins, RNA regulating translation, and RNA protein regulation, among others. As another example, nucleic fluid can provide a picture of potential mechanisms that may occur within a cell, mechanisms such as mutations. In yet another example, mitochondrial fluid can provide information as to the origin of the cell and the organism's matrilineal line.

While cellular analytics is useful, refinements to the operation may yield more detailed analysis results. For example, in general it may be difficult to obtain a correlation between 1) the mechanical and chemical properties of a cell and 2) the genetic information of the cell. That is, a user cannot simultaneously get mechanical and genetic information from a single sample. To get both genomic and mechanical information, two different samples would be used. However, as the different samples may have different properties, any correlation between the separately collected genomic and mechanical information would rely on a similarity between the two samples, which similarity may not exist or may be tenuous.

Accordingly, a scientist may have to pick from between the two pieces of information (e.g., mechanical and genomic), which they would like to collect. It may be more desirable to obtain the genomic information from the cell as it provides more information. However as described above, the mechanical properties of a cell also provide valuable information. For example, lysis information allows a user to infer cell mechanical properties which may indicate to the user the state of the cell, i.e., dead/living, diseased/healthy.

Moreover, in cellular analytics it may be desirable to know the correlation between a phenotype and a genotype of a cell. Information about this correlation may lead to a better understanding of chemical signaling pathways within the cell. Knowing the chemical signaling pathways allows for a greater understanding of cell function and response to stimuli. For example, a correlation between genomic information and a cells susceptibility to lysis may allow a prediction of lytic antibiotic resistance of a cell based on the cells' genetic information. Disease pathology is a specific example as mechanical properties play a particular role in disease pathology. For example, the elasticity (mechanical property) of a circulating tumor cell may be a determining factor of the cell's metastatic potential and therefore may be an indicator of cancerous cells. In this example, the genetic information collected form a sample indicates what mutations are activated in the cell and may indicate which pathways are up or down regulated. From the genetic and mechanical information, a medical professional may determine which chemotherapy to prescribe as the role of many chemotherapeutics is to affect these pathways. As yet another example, malaria, which is a parasitic infection of red blood cells that changes a stiffness (mechanical property) of the red blood cells and changes the transportation of these cells through the circulatory system. By obtaining the genetic information at the same time, a scientist may determine a type of parasite (there are many malarial parasites for example) that are affecting the patient. With such detailed solutions, a more specific anti-malarial process may be followed. Accordingly, both pieces of information, i.e., mechanical properties and genetic information, for a cell are valuable and useful in analytic chemistry.

Still further, many cell populations are heterogeneous, meaning each cell in a population may be different from others and may have different responses and characteristics. Accordingly, the correlation between mechanical and genetic information may also be heterogeneous. Accordingly, it may be desirable to obtain genomic and mechanical properties at a single cell level so as to remove inter-sample variation from any resulting correlation.

In some examples, cells may be marked such that they may be later sorted and analyzed. That is, a marker is a physical tag associated with a particular cell such that as the cell passes through a cellular analytic system, it may be tracked. The tracking of a cell through a cell analysis system provides an organization to the information collected. That is, it ensures that particular information collected during cellular analysis is associated with the appropriate cell.

While some solutions have been presented for identifying cells in a population, they are inadequate for any number of reasons. For example, cells may be sorted optically using a fluorescence activated cell sorting (FACS) operation. In this example, marking is done manually in a separate vessel, with an excess of marking compound. In this example, the marker may non-uniformly adhere to the cells. In this process, the cells are also exposed to atmosphere, which risks damage to the cells. Moreover, the systems that implement FACS are large, expensive, and do not lyse the cells.

This FACS process may take several hours with several manual operations. Cell lysis and any downstream analysis are therefore not correlated with the staining information, specifically on a single cell level. Moreover, as the time between sorting and lysing is long and certain biological cells may change over that period of time, any correlation that may be determined, is inconclusive and likely erroneous.

Accordingly, the present specification describes a system that provides automated single cell sorting within the same device that performs cell lysis. The present system applies a marker individually to single cells and differentiates cells based on their response to cell markers. As a particular example, using an antibody-based marking, the present system can differentiate cells based on certain surface antigens present on the surface of the cell.

As this all occurs on the same device, the time between sorting and lysing is very short, thus allowing the present system to be robust against the rapidly changing profiles of biological molecules inside the cell.

In other words, the present specification describes a cell marking system with optical detection. A cell analysis system in which the cell marking system is implemented tracks the cells following lysing and delivers the marked cells to a downstream analysis device.

In one example, the cell analysis system includes a precision staining inkjet arrangement for dispensing stain droplets onto cells or injecting stain into a flow path via integrated pumps. The system also includes optical detection, tracking systems, cell lysis elements, and ejection elements. Such a cell marking and analysis system automatically stains cells, sorts them based on their staining profile, lyses them, and ejects the lysate to individual compartments for downstream analysis.

Specifically, the present specification describes a cell marking system. The cell marking system includes a microfluidic channel to serially feed individual cells from a volume of cells into at least one marking chamber. The at least one marking chamber holds an individual cell to be marked and a marker application device per marking chamber selectively apples a marker to the individual cell disposed within a respective marking chamber.

The present specification also describes a method. According to the method, a quantity of cells from a cell reservoir is passed in serial fashion to at least one cell marking system of a microfluidic cell analysis system. For each cell marking system, it is determined whether a cell is to be marked. Also, per cell marking system, a marker is applied to selected cells. The marker remains on a cell membrane and changes at least one of an optical and electrical property of a selected cell.

The present specification also describes a cell analysis system. The cell analysis system includes at least one cell analysis device. Each cell analysis device includes the microfluidic channel, at least one marking chamber and marker application device per marking chamber. In this example, the cell analysis device also includes a detector to detect which cells have been marked. The at least one cell analysis device also includes a feedback-controlled lysing device that includes a lysing chamber and at least one lysing element in the lysing chamber to agitate the individual cell. The feedback-controlled lysing device also includes a sensor to detect a state within the lysing chamber. A controller of the cell analysis system analyzes the individual cell. The controller includes 1) a lysate analyzer to analyze properties of a lysate of the individual cell, 2) a rupture analyzer to analyze parameters of an agitation when a cell membrane ruptures, and 3) a component controller to activate components of the cell analysis system based on an output of the marker detector.

In summary, using such a cell analytic system 1) allows single cell analysis of a sample; 2) allows combined cell analysis, i.e., a genetic analysis and a mechanical property analysis; 3) can be integrated onto a lab-on-a-chip; 4) is scalable and can be parallelized for high throughput, 5) is low cost and effective; 6) reduces stain consumption; 7) allows tracking of a cell through a cell analysis system; 8) is robust against the rapidly changing profile of some cells; 9) accommodates different stains; 10) provides for real-time sample preparation; and 11) automates the cell preparation operation. However, the devices disclosed herein may address other matters and deficiencies in a number of technical areas.

As used in the present specification and in the appended claims, the term “cell membrane” refers to any enclosing structure of a cell, organelle, or other cellular particle.

Further, as used in the present specification and in the appended claims, the term “agitation cycle” refers to a period when a cell is exposed to the operations of a lysing element. For example, an agitation cycle may refer to each time a cell is looped past a single lysing element. In another example, a cell passes through an agitation cycle each time it passes by a lysing element in a string of multiple lysing elements.

Even further, as used in the present specification and in the appended claims, the term “rupture threshold” refers to the amount of stress that a cell can withstand before rupturing. In other words, the rupture threshold is the threshold at which the cell ruptures. The rupture threshold may be determined based on any number of factors including a number of agitation cycles a cell is exposed to and the intensity of the agitation cycles.

Yet further, as used in the present specification and in the appended claims, the term “parameters” refers to the operating conditions in a particular agitation cycle. For example, a “parameter” may refer to a type of lysing element and/or a lysing strength. For example, agitation parameters for an agitation cycle may include whether a lysing element is a thermal inkjet resistor, a piezo-electric device, or an ultrasonic transducer. Agitation parameters also refer to the operating conditions of the particular lysing element. For example, the parameters of an ultrasonic transducer may refer to the frequency, amplitude, and/or phase of ultrasonic waves. The parameters of the thermal inkjet resistor and piezo-electric device may refer to the size of the element and/or the voltage applied to the element.

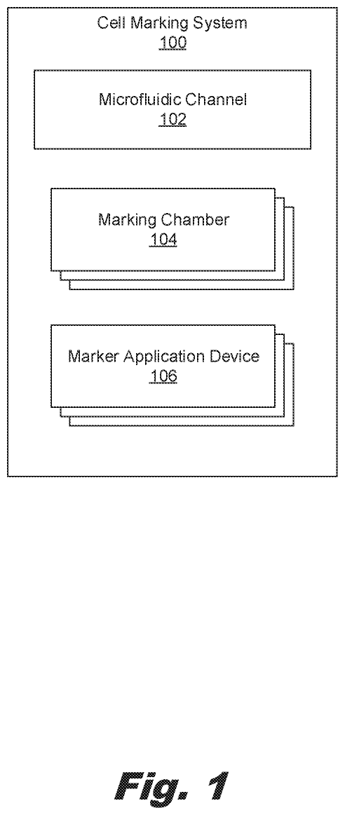

Turning now to the figures, FIG. 1 is a block diagram of a cell marking system (100), according to an example of the principles described herein. In some examples, the cell marking system (100) is part of a lab-on-a-chip device. A lab-on-a-chip device combines several laboratory functions on a single integrated circuit which may be disposed on a silicon wafer. Such lab-on-a-chip devices may be a few square millimeters to a few square centimeters, and provide efficient small-scale fluid analysis functionality.

In other words, the components, i.e., the microfluidic channel (102), marking chamber(s) (104), and marker application device(s) (104) may be microfluidic structures. A microfluidic structure is a structure of sufficiently small size (e.g., of nanometer sized scale, micrometer sized scale, millimeter sized scale, etc.) to facilitate conveyance of small volumes of fluid (e.g., picoliter scale, nanoliter scale, microliter scale, milliliter scale, etc.).

The microfluidic channel (102) delivers cells to the at least one marking chamber (104). Specifically, the microfluidic channel (102) passes the cells in individual fashion to the marking chamber(s) (104). That is, the cell marking system (100) of the present specification describes a per-cell marking. Accordingly, the microfluidic channel (102) may have properties such that cells are passed individually. Such a serial, single-file introduction of cells into the marking chamber (104) may be facilitated by microfluidic channels (102) having a cross-sectional area size on the order of the cell diameter. The microfluidic channel (102) is coupled at one end to a cell reservoir and directs cells single-file into a marking chamber (104).

The cell marking system (100) also includes at least one marking chamber (104) to hold a cell to be stained or marked. In some examples, the cell marking system (100) includes multiple marking chambers (106). In one example, the multiple marking chambers (104) are used to apply different markers to one cell. In another example, the multiple marking chambers (104) apply different markers to different cells. In yet another example, the multiple marking chambers (104) eject different agents in a multi-stage marking operation.

The marking chamber(s) (104) may be no more than 100 times a volume of a cell to be marked. In other examples, the marking chamber(s) (104) may have a cross-sectional size comparable with the cell size. That is, the marking chamber(s) (104) may be microfluidic structures.

As the marking chamber (104) is the location where marking occurs, the marking chamber (104) receives a cell or other component to be marked. As described above, the marking chamber (104) may receive the cells single-file, or serially. Thus, marking operations can be performed on a single cell and that cell's particular properties may be analyzed and processed.

A marker application device (106) applies the marker onto the cell. The marker application device (106) may take a variety of forms. For example, the marker application device (106) may be on a different physical structure and may eject the marker through an orifice in the marking chamber (104). That is, the marker application device (106) may be formed on a second substrate that is distinct from a first substrate on which the marking chamber (104) is formed. In another example, the marker application device (106) is integrated into a same substrate as the marking chamber (104) and may pump the marker into the marking chamber (104).

The marker that is applied may be of a variety of types. In general, the marker may be a stain that in one way or another enhances the contrast in a microscopic image. This may be done by altering any of a number of properties of the cell. For example, a marker may alter an optical property of the cell. Specifically, a fluorescence, absorption, or light scattering property of a cell may be altered. As a specific example, the marker may be a fluorescent stain. In this example, the stain is chemically attached to the cell to aid in the detection of a component such as a protein, antibody, or amino acid. In these examples, the stain may be a fluorescent molecule such as fluorophore. Other examples of fluorescent stains that may be used include ethidium bromide, fluorescein and green fluorescent protein. As a specific example, the fluorescent stain may, in the absence of DNA, be non-fluorescent, but in the presence of DNA, the stain fluoresces. In other words, the presence of a certain molecule, such as DNA, induces a fluorophore to emit. In the absence of DNA, the marker floats in water and interacts with dissolved oxygen and the oxygen quenches the marker and does not permit fluorescence. When DNA is present, the stain intercalates into the DNA molecule and is shielded from the oxygen such that no quenching takes place. In this example, the marker now fluoresces. This change can be detected and used for downstream analysis. Similarly, other optical properties such as absorption and light-scattering properties may be adjusted via chemical attachment of a particular staining agent.

The stain may also alter an electrical property such as a membrane capacitance. As will be described below, the change in property may be detected by a downstream detector and certain operations executed/prohibited based on the presence or absence of a marker.

As another specific example, the stain may be anti-body based. In one example an antibody is chemically labeled with a fluorophore molecule. This antibody is released into a solution with cells. The antibody is attracted to an antigen on the surface of the cell and binds to it. The cells are then observed with, for example, a fluorescence microscope. In another example, an unlabeled antibody is released into solution and similarly binds to cells. As a second step, another antibody which is fluorescently labeled, is introduced into the solution and binds with the first antibody. In this example, the first antibody serves as an antigen. Cells are again observed, for example under a fluorescence microscope. As a specific example, a user may desire to stain a leukocyte. Accordingly, a small amount of CD45 antibody that is combined with a die may be ejected, which adheres to the surface of the leukocyte.

The marker may be a one-stage marker or a multi-stage marker. By implementing multiple marking chambers (104), multi-stage marking may be accommodated. Multiple marking chambers (104) also facilitate application of different markers to target different cells based on a cell response. That is, certain cells may respond a certain way to a first marker and different cells may respond a certain way to a second marker. To differentiate the two, each cell may be marked by a distinct marker application device (106) with the respective marker.

In some examples, prior to introduction into the cell marking system (100), the cells may be treated. That is, the surface of the cells may be prepared to more readily accept an applied marker.

Examples of specific markers that may be used include acridine orange, carmine, ethidium bromide, safranine, crystal violet, and propidium iodide. While specific reference is made to a few particular markers, a variety of markers may be used in the cell marking system (100) as described herein.

Accordingly, the present specification describes a cell marking system (100) that is integrated with a microfluidic cell analysis system. Thus, marking occurs in the same structure as where cell lysis occurs, thus reducing exposure to environmental conditions and reducing the potential damage that may result therefrom. Moreover, by implementing microfluidic structures such as a microfluidic channel (102), single cell marking may be implemented which is a more precise method of cell marking as each cell is targeted. Thus, by single cell marking, the cell marking system (100) facilitates subsequent single cell analysis by providing a tracking mechanism for each cell through the cell analysis system.

Such a precise sorting mechanism provides a number of benefits. For example, sorting can be used to distinguish, differentiate, and detect. For example, given a population of blood cells and bacteria cells, such a cell marking system (100) allows for differentiation of the bacteria cells and blood cells such that the bacteria cells can be analyzed without the influence of the blood cells in the population.

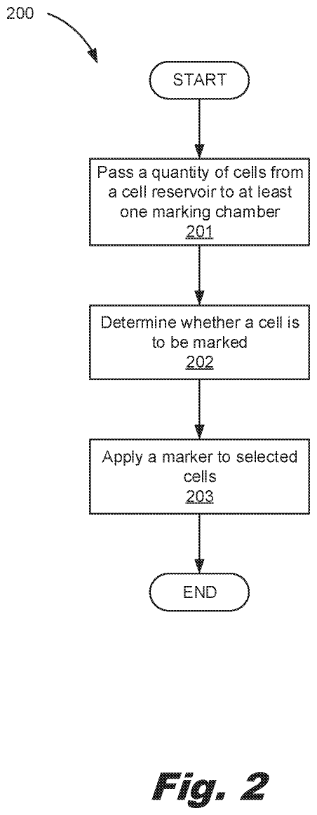

FIG. 2 is a flow chart of a method (200) of cell marking, according to an example of the principles described herein. In the method (200), a quantity of cells to be analyzed are passed (block 201) from a cell reservoir to at least one cell marking system (FIG. 1, 100). That is, the cell analysis system may include one, or multiple cell marking systems (FIG. 1, 100). Implementing multiple cell marking systems (FIG. 1, 100) facilitates increased throughput by parallelizing the operations of the cell marking systems (FIG. 1, 100). As described above, the cell marking system (FIG. 1, 100) may be a component of a microfluidic cell analysis system.

In some examples, the cells are serially passed (block 201) to each cell marking system (FIG. 1, 100). That is, each cell within the sample may be received (block 201) one at a time. In some examples, each cell marking system (FIG. 1, 100) includes a microfluidic channel (FIG. 1, 102) that gates introduction of one cell at a time into the marking chamber (FIG. 1, 104) for marking. Such single-file, or serial, inlet of cells facilitates an individual marking of cells. Accordingly, rather than marking a group of cells and hoping that particular cells are marked, individual cells can be treated such that it may be ensured that targeted cells receive the desired marker. Moreover, by individually targeting cells for marker reception, marker compound is preserved.

The subsequent operations may be performed per cell marking system (FIG. 1, 100). Once in a marking chamber (FIG. 1, 104), it may be determined (block 202) whether a cell is to be marked. In some cases, each cell to be analyzed downstream may be marked, while those cells not to be analyzed are not marked. Accordingly, in this example, the cell marking system (FIG. 1, 100) may include a cell presence sensor which activates the marker application devices (FIG. 1, 106). Thus, rather than expelling marker compound continuously, marker compound is ejected just when it is determined that a cell of interest is present. Thus, marking compound may be preserved.

The distinction of those cells to be marked and those not to be marked may be determined based on an output of the cell presence sensor. The cell presence sensor may be of any variety of types. That is, the cell presence sensor may be an impedance sensor, an optical scatter sensor, an optical fluorescence sensor, an optical bright field imaging system, an optical dark field imaging system, or a thermal property sensor. Such a sensor may distinguish cells based on different detected properties.

This cell presence sensor is disposed before the marking chamber (FIG. 1, 104) and may trigger activation of the marker application device (FIG. 1, 106). For example, if the cell presence sensor indicates that a cell is not present, a controller of the cell marking system (FIG. 1, 100) may avoid activating the marker application device (FIG. 1, 106). By comparison, if the cell presence sensor sends indicates that a cell is present, the controller may activate the marker application device (FIG. 1, 106). As described above, the cell presence sensor may not only detect whether a cell is present, but whether the cell is of a type intended to be marked.

In one particular example, the cell presence sensor is an impedance sensor. Specifically, the cell presence sensor may include at least one pair of electrodes spaced apart from one another by a gap. These electrodes detect a level of conductivity within the gap. That is, incoming cells to a marking chamber (FIG. 1, 104), and the solution in which they are contained, have a predetermined electrical conductivity. Different cells have a different electrical conductivity. If the conductivity between the electrodes maps to a cell to be marked, the system applies (block 203) a marker to the selected cell. By extension, if the conductivity between the electrodes does not map to a cell to be a marked, the system does not apply the marker to that cell. As described above, the marker changes at least one of an optical or electrical property of the cell such that cells of interest may be distinguished from other cells throughout the cell analysis system.

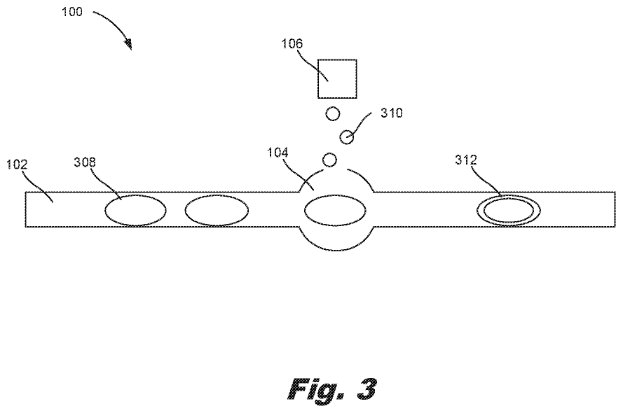

FIG. 3 is a diagram of a cell marking system (100), according to an example of the principles described herein. FIG. 3 depicts the microfluidic channel (102) that routes the cells throughout the cell marking system (100) and that routes the cells throughout the larger cell analysis system. FIG. 3 also depicts the cells as they pass through the channel (102). Specifically, FIG. 3 depicts the unmarked cells (308) entering into the marking chamber (104) and the marked cells (312) that pass out of the marking chamber (104) to downstream devices such as a lysing chamber. As described above, the cells (308, 312) may be passed single-file through the microfluidic channel (102) such that each is individually marked, lysed, and ejected. FIG. 3 also depicts the marking chamber (104) and the marker application device (106). As described above, in some examples the marker application device (106) may be external to the marking chamber (104). Specifically, the marker application device (106) is on a second substrate that is distinct from a first substrate on which the respective marking chamber (104) is formed. In this example, the marking chamber (104) includes an orifice through which the marker (310) is received into the marking chamber (104) and ultimately deposited on the cell disposed within the marking chamber (104).

The marker application device (106) may be a firing resistor or other thermal device, a piezoelectric element, or other mechanism for ejecting fluid from the firing chamber. For example, the marker application device (106) may be a thermal inkjet ejector that ejects the marker (310) into the respective marking chamber (104). The thermal inkjet ejector includes a firing resistor. The firing resistor heats up in response to an applied voltage. As the firing resistor heats up, a portion of the marker (310) in the marker application device (106) vaporizes to form a bubble. This bubble pushes the marker (310) out the opening and through the orifice into the marking chamber (104). As the vaporized fluid bubble collapses, a vacuum pressure along with capillary force draws marker (310) into the marker application device (106) chamber from a reservoir, and the process repeats. In this example, the marker application device (106) may be a thermal inkjet ejector.

In another example, the marker application device (106) may be a piezoelectric device. As a voltage is applied, the piezoelectric device changes shape which generates a pressure pulse in the firing chamber that pushes a fluid out the opening. In this example, the marker application device (106) may be a piezoelectric inkjet ejector.

Specifically placing the marker (310) on the cell increases marker efficiency. That is, applying the marker (310) in direct proximity to the cell minimizes marking time, increases marker uniformity and reproducibility as the reliance on diffusion and mixing to deliver the marker (310) is reduced.

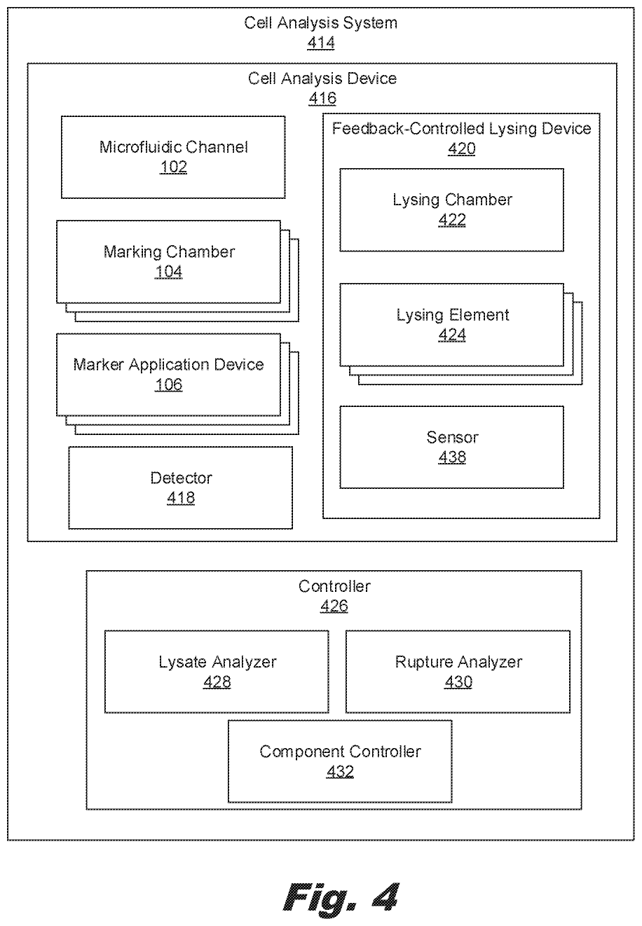

FIG. 4 is a block diagram of a cell analysis system (414), according to an example of the principles described herein. In some examples, the cell analysis system (414) is part of a lab-on-a-chip device. A lab-on-a-chip device combines several laboratory functions on a single integrated circuit which may be disposed on a silicon wafer. Such lab-on-a-chip devices may be a few square millimeters to a few square centimeters, and provide efficient small-scale fluid analysis functionality.

In other words, the components, i.e., the cell analysis device(s) (418), microfluidic channel(s) (102), marking chamber(s) (104), detector (418), and feedback-controlled lysing device (420) may be microfluidic structures. A microfluidic structure is a structure of sufficiently small size (e.g., of nanometer sized scale, micrometer sized scale, millimeter sized scale, etc.) to facilitate conveyance of small volumes of fluid (e.g., picoliter scale, nanoliter scale, microliter scale, milliliter scale, etc.).

The cell analysis system (414) include at least one cell analysis device (416). The cell analysis device (416) refers to the components that perform multiple operations on a cell. In some examples, each component that makes up the cell analysis device (416) is disposed on a single substrate. Thus, each operation may be carried out on a single silicon substrate. That is, the present cell analysis system (414) facilitates the complete analysis of a cell, at a single cell resolution, on a single physical structure.

In other examples, different components may be on different substrates. For example, the marker application device (106) may be on a different substrate as depicted in FIG. 3. Also, as depicted in later figures, the detector (418) may be on a different substrate.

In some examples, the cell analysis system (414) may include multiple cell analysis devices (416) such that high cell throughput is attained. The substrate may be formed of any material including plastic and silicon, such as in a printed circuit board. The cell reservoir may be any structure that holds a quantity of cells to be analyzed.

The cell analysis device (416) includes the microfluidic channel (102) that delivers cells to the marking device (104). The microfluidic channel (102) also delivers cells to other components of the cell analysis device (416). The cell analysis device (416) also includes the marking chamber(s) (104) and marker application device(s) (106) as described above.

In this example, the cell analysis device (416) includes additional components. Specifically, the cell analysis device (416) includes a detector (418) to detect which cells have been marked. The detector (418) may be downstream of the at least one marking chamber (104) to detect which cells have been marked. In an example, an output of the detector (418) selectively activates a particular feedback-controlled lysing element (424).

That is, as described above, the marker (FIG. 3, 310) may alter an optical and/or electrical property of a particular cell and a detector (418) is a component that can detect such alteration. That is, the detector (418) can determine a fluorescence of a particular cell and can determine, based on a difference between a known fluorescence of an unmarked cell (FIG. 3, 308) can identify that the cell has been marked.

In one example, the detector (418) is a spectrometer. The spectrometer includes a grating to select a wavelength of light from a light source such as a mercury or xenon lamp. The fluorophore on the cells emits light that passes through another grating, which directs a particular frequency of light onto a charge-coupled device (CCD) array. In this example, the gratings may move and the angle of the grating relative to the angle of the incoming light selects a wavelength of interest.

In some examples, an output of the detector (418) may selective activate a particular lysing element (424). That is, the cell analysis system (414) may include various feedback-controlled lysing devices (420) each to lyse a different type of cell. Each cell may be differentiated from one another based on 1) whether it is marked and/or 2) the type of marking. When an output of the detector (418) indicates a marking associated with a particular cell to be lysed, the corresponding lysing element (424) may be activated to lyse that cell while other lysing elements (424) remain inactive. Such a cell-based lysis activation conserves power as the lysing element (424) is deactivated at times it is not needed.

In addition to activating a particular lysing element (424), the marker response of a cell, as detected by the detector (418), may trigger other actions. For example, based on a marker response of a marked cell, the detector (418) may trigger activation of a particular pump to draw a marked cell into a particular branched channel where a corresponding lysing element (424) resides. Similarly, based on a marker response of a marked cell, the detector (418) may activate a waste ejector to eject the unmarked cell from the cellular analytic system (414) in which the cell marking system (FIG. 1, 100) is disposed.

In other words, the detector (418) can detect the presence of a marked cell based on changes to the property that is altered by the marker (FIG. 3, 310). Based on the properties of the marker response, i.e., the response to the marker (FIG. 3, 310), the detector (418) triggers any number of actions that depend on the properties of the detected cell.

The cell analysis device (416) also includes a feedback-controlled lysing device (420). In general, lysis refers to the agitation of a cell with the objective of rupturing a cell membrane. Lysis ruptures a cellular particle membrane and frees the inner components. The fluid containing the inner components is referred to as lysate. The contents of the cellular particle can then be analyzed by a downstream system.

The feedback-controlled lysing device (420) includes a lysing chamber (422) where lysing and lysis detection occur. In some examples the lysing chamber (422) may be no more than 100 times a volume of a cell to be lysed. In other examples, the lysing chamber (422) may have a cross-sectional size comparable with the cell size and in some cases smaller than the cell so as to deform the cell before or during the rupturing of the cell membrane. That is, the lysing chamber (422) may be a microfluidic structure.

As the lysing chamber (422) is the location where lysis occurs, the lysing chamber (422) receives a cell or other component to be lysed. In some examples, the lysing chamber (422) may receive the cells single-file, or serially. Thus, lysing operations can be performed on a single cell and that cell's particular properties may be analyzed and processed.

In some examples, the lysis operation may be feedback-controlled. Accordingly, the lysing chamber (422) includes a lysing element (424) to carry out such an agitation and a sensor (438) to detect a state within the lysing chamber (422). The lysing element (424) may implement any number of agitation mechanisms, including shearing, ball milling, pestle grinding, and using rotating blades to grind the membranes. Other examples of agitation mechanisms include localized heating and shearing by constriction. In another example, repeated cycles of freezing and thawing can disrupt cells through ice crystal formation. Solution-based lysis is yet another example. In these examples, the osmotic pressure in the cellular particle could be increased or decreased to collapse the cell membrane or to cause the membrane to burst. As yet another example, the cells may be forced through a narrow space, thereby shearing the cell membranes.

In one example, the lysing element (424) is a thermal inkjet heating resistor disposed within the lysing chamber (422). In this example, the thermal inkjet resistor heats up in response to an applied current. As the resistor heats up, a portion of the fluid in the chamber vaporizes to generate a bubble. This bubble generates a pressure and shear spike which ruptures the cell membrane.

In another example, the lysing element (424) may be a piezoelectric device. As a voltage is applied, the piezoelectric device changes shape which generates a pressure pulse in the chamber that generates a pressure and shear spike which ruptures the cell membrane.

In yet another example, the lysing element (424) may be a non-reversible electroporation electrode that forms nano-scale pores on the cell membrane. These pores grow and envelope the entire cell membrane leading to membrane lysis. In yet another example, the lysing element (424) is an ultrasonic transducer that generates high energy sonic waves. These high energy waves may travel through the wall of the chamber to shear the cells disposed therein.

The different types of lysing elements (424) each may exhibit a different agitation mechanism. For example, the agitation mechanism of an ultrasonic transducer is the ultrasonic waves that are emitted and that shear the cells. The agitation mechanism of the thermal inkjet heating resistor is the vapor bubble that is generated and ruptures the cell membrane. The agitation mechanism of the piezo-electric device is the pressure wave that is generated during deformation of the piezo-electric device, which pressure wave shears the cell membrane. While particular examples of lysing elements (424) have been described herein, a variety of lysing element (424) types may be implemented in accordance with the principles described herein.

A feedback-controlled lysis operation refers to a lysis operation that is monitored to ensure lysis occurs as desired. That is, the feedback provides a quality control check over a lysing operation. In this example, the lysing chamber (422) includes a sensor (426) to determine when a cell has ruptured, and to return the cell to within range of the feedback-controlled lysing element (424) in the case the cell has not ruptured. That is, the sensor (438) detects a change in the cell based on an agitation of the cell by the at least one lysing element (424). If no change is detected, the cell is kept in, or returned to, the lysing chamber (422) for another agitation cycle. Accordingly, rather than activating the lysing element (424) and hoping that lysing occurs, a lysing device (420) includes a sensor (438) to ensure lysing occurs prior to further processing of the lysate.

In some examples, the cell analysis device (416) gradually increases the intensity of agitation such that it can be precisely determined at what stress level a particular cell ruptures. Increasing the agitation intensity may include increasing the intensity of the lysing element (424) and/or by increasing a count of how many exposures the cell has to the lysing element (424). For example, a lysing element (424) intensity may not change, but the cell may be passed by the lysing element (424) multiple times until cell rupture occurs. In another example, a lysing element (424) intensity increases and the cell may be passed by the lysing element (424) multiple times until cell rupture occurs.

The cell analysis system (414) also includes a controller (426) that analyzes the cells of the sample. The controller (426) includes various components to make such an analysis. First, the controller (426) includes a lysate analyzer (428) to receive information regarding the lysate. That is, after the cell has been ruptured, the contents therein may be analyzed and information provided to the lysate analyzer (428). A variety of pieces of information can be collected from the lysate. For example, cytoplasmic fluid within the cell may provide a picture of the current mechanisms occurring within the cell. Examples of such mechanisms include ribonucleic acid (RNA) translation into proteins, RNA regulating translation, and RNA protein regulation, among others. As another example, nucleic fluid can provide a picture of potential mechanisms that may occur within a cell, mechanisms such as mutations. In yet another example, mitochondrial fluid can provide information as to the origin of the cell and the organism's matrilineal line.

The controller also includes a rupture analyzer (430) which determines a rupture threshold of the cell based on the parameters of the agitation when the cell membrane ruptures. That is, as described above a cell may be exposed to one or multiple agitation cycles. Information regarding the parameters (type, strength, and count) of the agitation cycles are passed to the rupture analyzer (430) which determines a rupture threshold of the cell based on the parameters of the agitation when the cell membrane ruptures. That is, as described above a cell may be exposed to gradually increasing intensities of lysing operations. The characteristics of the different agitation cycles can be passed to the controller (426) which determines a rupture threshold.

The parameters of the different agitation cycles can be passed to the rupture analyzer (430) which determines a rupture threshold. The rupture analyzer (430) may use this information to perform a variety of analytical operations. For example, the rupture analyzer (430) may differentiate cells in a sample based on different rupture thresholds. In this example, the rupture analyzer (430) may receive, for multiple cells, information regarding the results of lysing by different lysing elements (426) on those cells. Based on the results, the rupture analyzer (430) may determine when each cell in a sample is ruptured. Different types of cells may rupture under different intensities. Accordingly, based on when a cell ruptures, the rupture analyzer (430) may be able to determine the cell types of the various cells in a sample.

As another example, the rupture analyzer (430) may be able to determine a state of a cellular sample. For example, it may be determined that healthy cells rupture at a particular lysing intensity. This may be determined by passing healthy cells through the cell analysis system (414) and collecting rupturing information. Accordingly, a sample to be analyzed may subsequently be passed through the cell analysis system (414) and rupturing information collected for these cells in the sample. If the rupturing information indicates that the sample cells rupture at a lower intensity than the healthy cells, the rupture analyzer (430) may determine that the sample cells are diseased.

As yet another example, the rupture analyzer (430) may be able to differentiate between live cells and dead cells based on the rupturing thresholds of different cells as determined by the cell analysis device (416). That is, live cells may be more robust against lysing and therefore have a higher rupturing threshold as compared to dead cells which may rupture at a lower intensity.

Thus, the present cell analysis system (414) provides a way to collect information related to both the lysate and the mechanical properties of the cell membrane from a single sample. Being able to collect both pieces from a single sample removes any bias resulting from intra-sample variation. For example, both the elasticity of a circulating tumor cell as well as the genetic components of the tumor cell may be determined from a single sample. As yet another example, both a stiffness of a red blood cell as well as the genetic aspects of the cell can be analyzed to determine if the cell is affected by malaria. Being able to collect both pieces of information from a single sample also makes more effective use of the sample. That is, rather than requiring two groups of the sample, one for mechanical testing and one for genetic testing, both pieces of information from one group of the sample.

The controller (426) also includes a component controller (432) to activate components of the cell analysis system (414) based on an output of the detector (418). For example, the component controller (432) may independently activate/deactivate certain of the lysing elements (424) and associated pumps. For example, a particular lysing element (424) may be activated/deactivated based on detection of a particular marker. That is a particular marker (FIG. 3, 310) may identify a particular type of cell for which the lysing element (424) is particularly intended to operate upon. Accordingly, when this particular marker (FIG. 3, 310) is detected, the lysing element (424) may be activated to lyse that cell and a pump adjacent the lysing element (424) may be activated to draw the cell towards that lysing element (424). Based on the detection of different markers (FIG. 3, 310), the component controller (432) may activate different lysing element (424)/pump pairs. That is, the detector (418) can distinguish cells based on a particular marker response and each marker response identifies a cell as a particular type and a corresponding lysing element (424)/pump pair that is to lyse that particular type of cell may be activated based on the output of the detector (418).

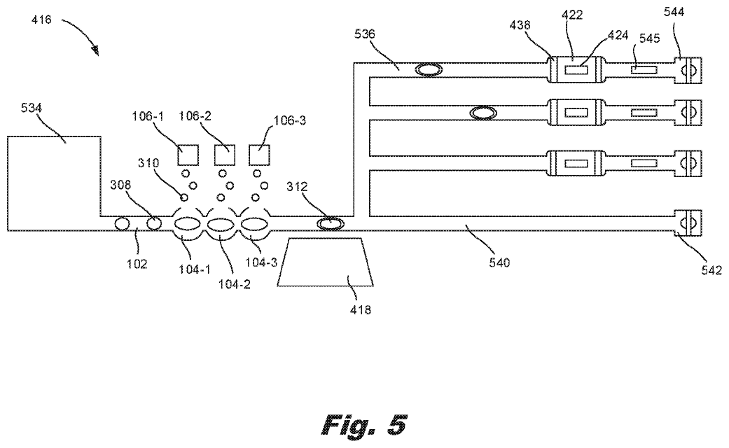

FIG. 5 is a diagram of a cell analysis device (416), according to an example of the principles described herein. As described above, the cell analysis system (FIG. 4, 414) includes at least one cell analysis device (416) which performs the cellular analysis. In some examples, a single cell analysis device (416) is used in the cell analysis system (FIG. 4, 414). However, the cell analysis system (FIG. 4, 414) may include multiple cell analysis devices (416), each to analyze an individual cell. In this example, the multiple cell analysis devices (416) may be in parallel. The multiple parallel cell analysis devices (416) facilitate the processing of more cells.

First, as described above the cell sample may be retained in a cell reservoir (534), which may be any container or receptacle to hold a sample of cells to be analyzed by the cell analysis device (416). The cell reservoir (534) may be coupled to each of multiple cell analysis devices (416).

In this example, prior to passing to the lysing chambers (422) where the cell is to be agitated, the cells in the sample may be sorted. Specifically, the sorting system differentiates cells based on their response to a marker (310) applied thereto. For example, a particular sample may include a variety of cells, but a single type of cell may be desired to be analyzed by the cell analysis system (FIG. 4, 414). Accordingly, the sorting system separates the desired cell to be analyzed from other cells in the sample and/or the carrier fluid of the sample. Doing so provides a more concentrated solution of the cells.

Moreover, by excluding undesirable cell types from being analyzed, any results are more particularly mapped to the desired cell. That is, the results of an analysis of a particular cell would not be skewed by analysis of a disparate cell type. As yet another example, the sorting of the cells, and in this case the marking of the cells, allows for results of cell analysis to be more clearly mapped to the original cells in the sample.

FIG. 5 depicts the microfluidic channel (102) that delivers unmarked cells (308) to the at least one marking chamber (104). In the example depicted in FIG. 5, the cell analysis device (416) includes three distinct marking chambers (104-1, 104-2, 104-3) and three corresponding marker application devices (106-1, 106-2, 106-3) which happen to be external, that is on a separate structure.

In this example, the different marker application devices (106) may eject different markers. That is, different markers (310) may be used to mark different cells such that the cells may be analyzed distinctly downstream. The different markers (310) may be applied to the same type of cell or different types of cells. For example, a first marker (310) may be applied to a first cell of a certain cell type via the first marker application device (106-1). A second marker (310) may be applied to a second cell of the certain cell type via the second marker application device (106-2). In this example, the cells may have different responses to the different markers (310). The different responses may be detected by the detector (418). The different markers (310) therefore may trigger activation of different pumps (545) to draw the differently marked cells to different lysis chambers (422) which different lysis chambers (422) may perform different (i.e., different strength) lysing operations.

In another example, the different markers (310) may be applied to different cell types. Similarly, the different responses may be detected by the detector (418). The different markers (310) therefore may trigger activation of different pumps (545) to draw the differently marked cells to different lysis chambers (422) which different lysis chambers (422) may perform different (i.e., different strength) lysing operations. The ability to mark cells differently provides for even more analysis paths as particular branched channels (536) may be particularly tailored for particular cells of a sample or to perform lysing operations of particular strength. That is, the cell analysis system (FIG. 4, 414), and particularly the cell analysis devices (416) include a number of branched channels (536). In this example each marked cell (312) is directed to a particular branched channel (536) based on a marker response associated with that cell. As an example, each of the branched channels (536) may perform unique/different lysing operations and the ability to differentiate between cells via the multiple marking chambers (104) allows for the direction of different cells to different of the branched channels (536) to make use of the different lysing operations. Thus, in general, using multiple markers (310) can provide better differentiation between different cells of a sample.

As another particular example, one marker (310) may be a stain and a second marker (310) may be a counterstain. A counterstain is a stain that has a color contrasting the primary stain. Thus, the primarily stained structure is more easily viewed.

One particular example of a differential stain is a gram stain which may be used to classify bacteria into two broad categories according to their cell wall. The gram status of a cell is relevant in medicine as the presence or absence of a cell wall changes the cell's susceptibility to some antibiotics. In general, the cell wall is rich in peptidoglycan and lacks a secondary membrane and lipopolysaccharide. In gram staining, those cells that are gram positive are stained one color and those that are gram negative are stained another color. This may be because of the presence of a thick layer of peptidoglycan on the cell walls alters stain absorption.

To perform gram staining, individual cells are introduced into the first marking chamber (104-1) and a particular marker (310), such as hexamethyl pararosaniline chloride is applied. In a second marking chamber (104-2), an iodine solution, for example of iodine and potassium iodide is added to form a complex between the hexamethyl pararosaniline chloride and iodine.

A counterstain, such as a weakly water-soluble safranin may then be applied via a third marking chamber (104-3). Since the safranin is lighter than the hexamethyl pararosaniline chloride it does not disrupt the purple coloration of the gram positive cells, however the decolorized gram negative cells are stained red.

FIG. 5 also depicts the detector (418) that is used to differentiate the cells based on their marker response. The detector (418) may be any type of detector that detects an alteration to the marked cells (310) based on the operation of the marker (310). That is, the marker (310) may alter any optical and/or electrical property of the marked cell (312) and the detector (418) can sense such an alteration. Accordingly, the detector (418) may be selected based on the type of marker (310) used and the alteration that marker (310) makes to the cell. As described above, the detector (418) output triggers certain downstream components. For example, the cell analysis device (416) may include any number of branched channels (536). For simplicity, a single instance of a branched channel (542), and the components therein, is identified with a reference number.

Each branched channel includes a lysing chamber (422) and a lysing element (424). The lysing elements (424) may be configured or designed to lyse with a particular strength or to be of a particular type to analyze a particular cell. Accordingly, when that particular cell is identified by the detector (418) based on its marker response, the respective lysing element (424) is activated as is a pump (545) that draws the cell towards that lysing element (424). As described above, different lysing elements (424) and corresponding pumps (545) are activated based on differently detected markers (310).

In some examples, the disparate cells and/or carrier fluid is ejected to a waste channel (540) that collects byproducts of the sorting. That is, cells not desired to be analyzed, i.e., unmarked cells, are passed to a waste channel (540) and ejected via a waste ejector (542).

FIG. 5 also depicts the sensor (438) used to determine whether the cell membrane was ruptured. The sensor (438) may take many forms. For example, the sensor (438), like the cell presence sensor may be an optical scatter sensor, an optical fluorescence sensor, an optical bright field sensing system, an optical dark field sensing system, a thermal property sensor, or an impedance sensor.

FIG. 5 also depicts the ejector (544) that expels the lysate. That is, each cell analysis device (416) includes an ejector (544) to eject the lysate. The lysate may be expelled by the ejector (544) to a downstream analysis device for further analysis.

Like the marker application devices (106), the ejector (544) may include a firing resistor or other thermal device, a piezoelectric element, or other mechanism for ejecting fluid from the firing chamber.

In some examples, the downstream analysis device may be a component of the cell analysis system (FIG. 4, 414) and/or device (416). That is, the downstream analysis device may be formed in the same silicon substrate as the other components, albeit in a different chamber. In yet another example, the downstream analysis device may be a separate component, for example a well plate to which the lysate is ejected.

In either case, information from the downstream analysis device and from the lysing element (424) is passed to a controller (FIG. 4, 426) for analysis and processing. That is, the controller (FIG. 4, 426) receives multiple types of information, 1) i.e., genomic/lysate information and 2) rupturing information from which a detailed cell analysis can be executed.

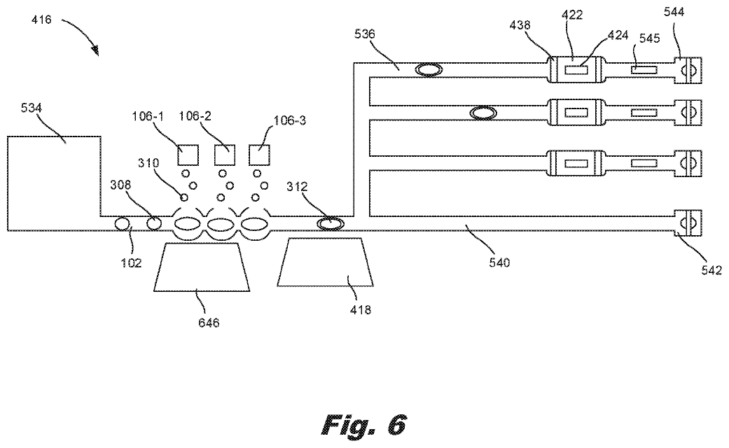

FIG. 6 is a diagram of a cell analysis device (416), according to another example of the principles described herein. In the example depicted in FIG. 6, the cell marking system (FIG. 1, 100) includes the cell presence sensor (646) to detect the presence of a cell to be marked. The cell presence sensor (646) may trigger activation of the marker application devices (106) based on a detected presence of the cell to be marked as described above. As described above, this cell presence sensor (646) is disposed before the marker application devices (106). If the cell presence sensor (646) sends information to the controller (FIG. 4, 426) which indicates that an unmarked cell (308) is not present, the component controller (FIG. 4, 432) may avoid activating the marker application devices (106). By comparison, if the cell presence sensor (646) sends information to the controller (FIG. 4, 426) which indicates that an unmarked cell (308) is present, the component controller (FIG. 4, 432) may activate the marker application device (106). By so doing, the cell analysis device (416) preserves marker (310) as marker (310) is not continually, or haphazardly ejected, but ejected at times when a cell is known to be positioned within the marking chambers (FIG. 1, 106). Doing so also ensures that marker (310) is completely and uniformly distributed over the cell to be marked.

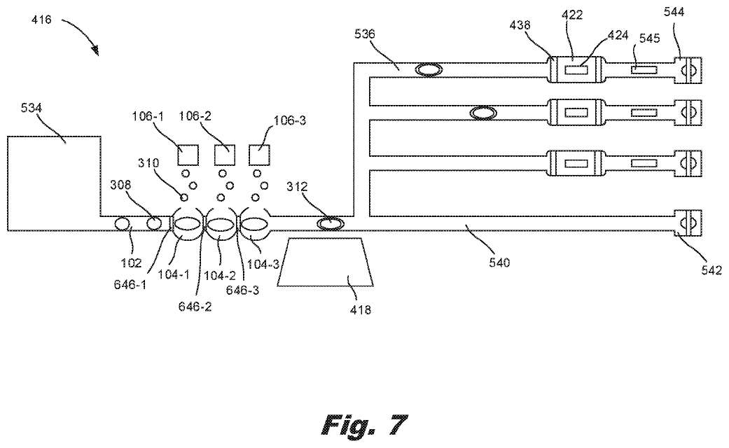

FIG. 7 is a diagram of a cell analysis device (416), according to another example of the principles described herein. FIG. 7 depicts a cell analysis device (416) similar to FIG. 6. However, FIG. 6 depicts an off-board cell presence sensor (646) while FIG. 7 depicts cell presence sensor(s) (646) that are on the same substrate as the other components. Specifically, FIG. 7 depicts an example where the cell presence sensors (646-1, 646-2, 646-3) are impedance sensors disposed within each marking chamber (104). In this example, rather than relying one cell presence sensor (646) to trigger each marker application device (106), the cell analysis device (416) may include multiple sensors (646) each paired with a particular marker application device (106) such that each marker application device (106) is individually triggered by the presence of a cell in a corresponding marking chamber (104).

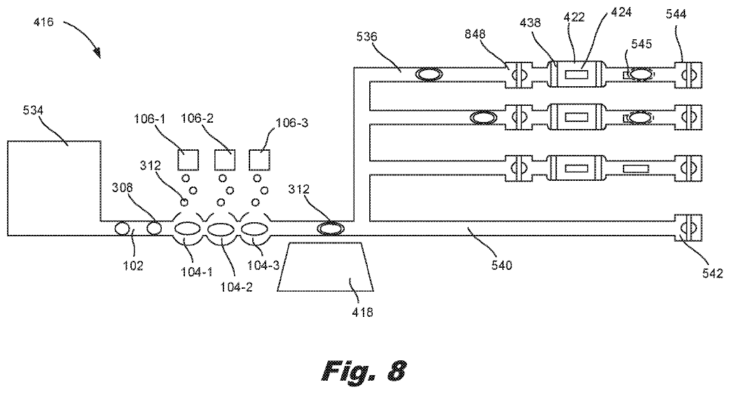

FIG. 8 is a diagram of a cell analysis device (416), according to another example of the principles described herein. The example depicted in FIG. 8 includes similar components described above. FIG. 8 also depicts a waste ejector (848) per branched channel (536) to eject unmarked cells from the particular branched channel (536). In this example, in addition to the waste channel (540) and waste ejector (542) coupled to the end of the waste channel (540), the waste ejector (848) provides an additional mechanism that removes waste fluid around the marked cells (312) to be analyzed. That is, in some cases, the cells to be analyzed may be rather dilute. The additional mechanism for removing waste fluid and/or unmarked cells increases the concentration of the cells to be analyzed, thus removing variability from any analysis operation. In the example depicted in FIG. 8, the waste ejector (848) in the branched channels (536) are before the lysis chamber (422) such that no waste fluid passes through the lysis chamber (422).

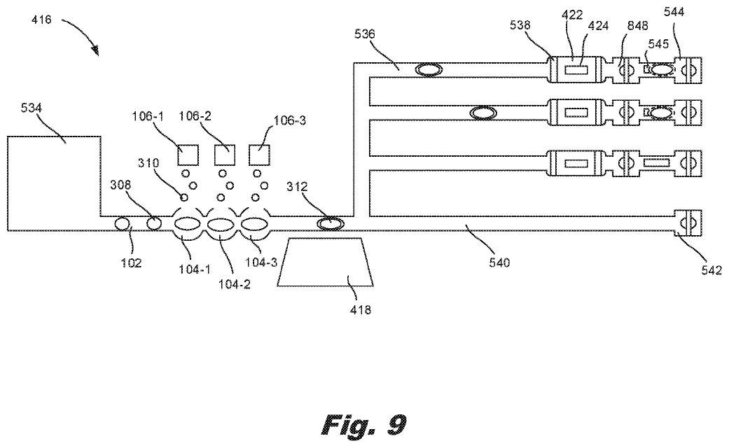

FIG. 9 is a diagram of a cell analysis device (416), according to another example of the principles described herein. FIG. 9 is similar to FIG. 8, with the exception that the waste ejector (848) per branched channel (536) is disposed immediately after the lysis chamber (422) and before the lysate ejector (544).

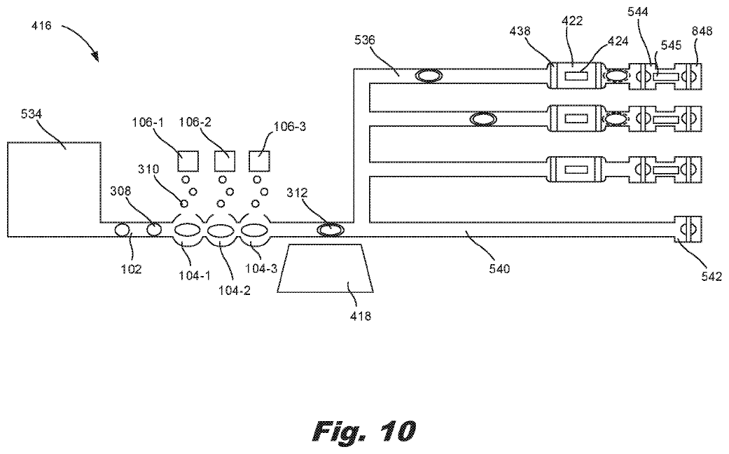

FIG. 10 is a diagram of a cell analysis device (416), according to another example of the principles described herein. FIG. 10 is similar to FIG. 9, with the exception that the waste ejector (848) per branched channel (536) is disposed downstream of the ejector (544) that ejects the lysate.

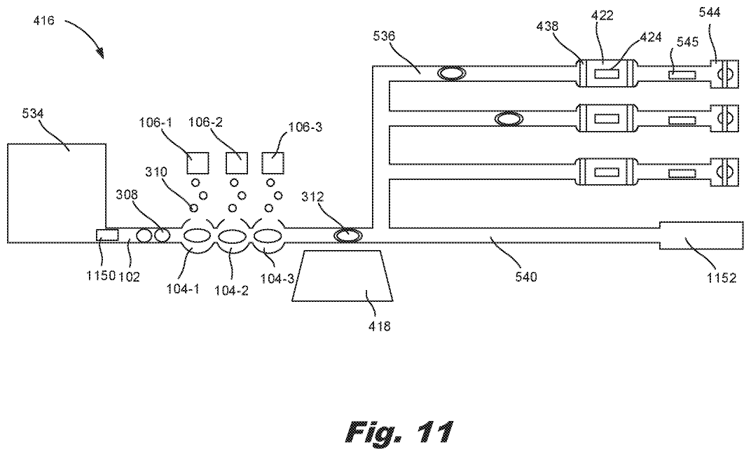

FIG. 11 is a diagram of a cell analysis device (416), according to another example of the principles described herein. In this example, the cell analysis device (416) includes an integrated pump (1150) disposed in the microfluidic channel (102) to move cells through the cell marking system. The integrated pump (1150), like the pumps (545), may be integrated into a wall of the microfluidic channel (102). In some examples, the pump (1150) may be an inertial pump which refers to a pump (1150) which is in an asymmetric position within the microfluidic channel (102). The asymmetric positioning within the microfluidic channel (102) facilitates an asymmetric response of the fluid to the pump (1150). The asymmetric response results in fluid displacement when the pump (1150) is actuated. In some examples, the pump (1150) may be a thermal inkjet resistor, or a piezo-drive membrane or any other displacement device.

FIG. 11 also depicts an example where the cell analysis device (416) includes a waste reservoir (1152). That is, rather than ejecting the waste fluid, the waste fluid is collected. In some examples, the waste reservoir (1152) is disposed on the substrate in which the marking chambers (104), lysis chamber (422) and other components are disposed. In this example, the waste fluid received in the waste reservoir (1152) includes unmarked cells and surrounding fluid.

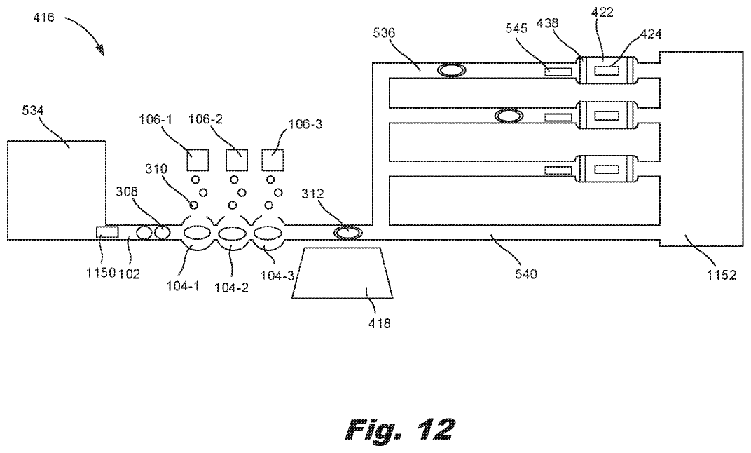

FIG. 12 is a diagram of a cell analysis device (416), according to another example of the principles described herein. FIG. 12 is similar to FIG. 11 with the exception that FIG. 12 depicts a shared waste reservoir (1152). The waste reservoir (1152) in FIG. 12, not only collects waste fluid that includes unmarked cells, but also includes waste fluid that may pass to each of the branched channels (536).

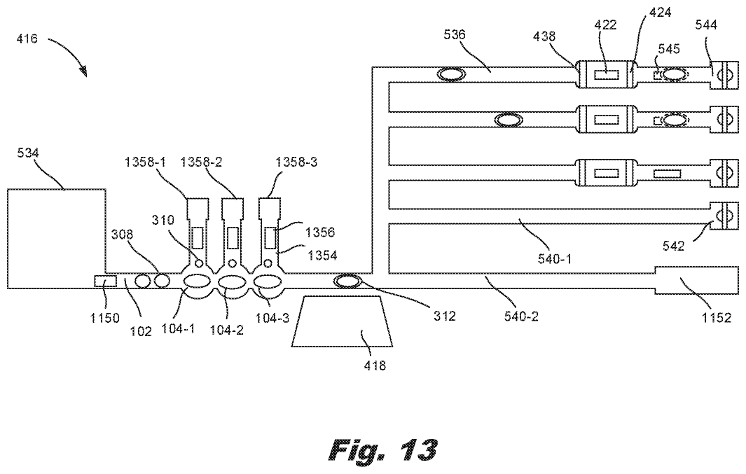

FIG. 13 is a diagram of a cell analysis device (416), according to another example of the principles described herein. As described above, in some examples the marker application devices (FIG. 1, 106) are located on a different substrate from the substrate in which the marking chambers (104) and lysing chambers (422) are formed. However, in other examples, the marker application devices (FIG. 1, 106) are formed on the same substrate. That is, the marker application devices (FIG. 1, 106) are fluidly coupled to the respective marking chamber (104) via a marking channel (1354). For simplicity, one marking channel (1354) is represented with a reference number. That is, in one example, the cell analysis device (416) includes a marker application device (FIG. 1, 106) in the form of a pump (1356) disposed in the marker channel (1354), which marker channel (1354) is formed on a same substrate on which the respective marking chamber (104) is formed.

In this example, as the marker (310) passes through an enclosed marker channel (1354), exposure to atmosphere is prevented, thus preserving the integrity and cleanliness of the system.

In this example, the cell marking system (FIG. 1, 100) also includes a marker reservoir (1358-1, 1358-2, 1358-4) to hold a volume of marker compound. The pump (1356) disposed in the marking channel (1354) transports the marker (310) from the marker reservoir (1358) into the marking chamber (104) and onto the cell.

FIG. 13 also depicts an example where the cell analysis device (416) includes a first waste channel (540-1) to direct waste fluid to a waste ejector (542) and a second waste channel (540-2) to direct waste fluid to a waste reservoir (1152). In this example, both the waste reservoir (1152) and the waste ejector (542) provide for the removal of waste fluid prior to lysis. The additional waste removal operation improves the waste removal process such that the concentration of cells to be analyzed and the resulting lysate is increased.

Note that any of the various combinations depicted in the different figures may be combined. For example, FIG. 13 depicts a waste reservoir (1152) that is not coupled to each branched channel (536). However, the example depicted in FIG. 13 with the integrated pumps (1356) acting as the marker application devices (FIG. 1, 106) may also implement the shared waste reservoir (1152) as depicted in FIG. 12.

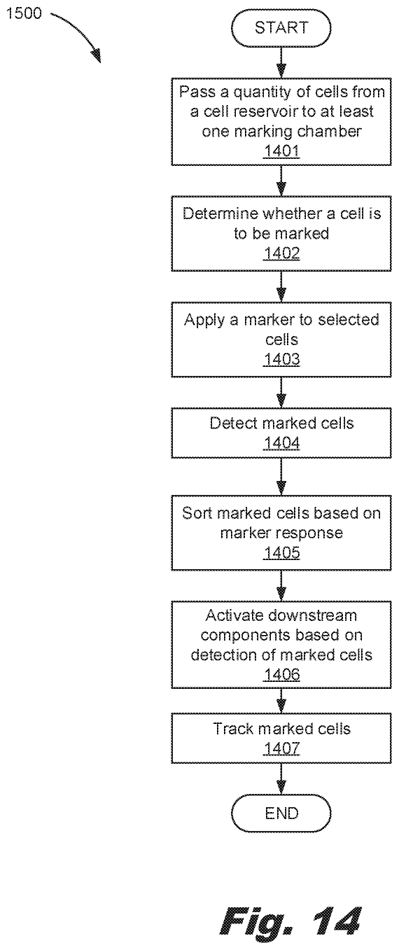

FIG. 14 is a flow chart of a method (1400) of cell marking, according to an example of the principles described herein. According to the method (1400) a quantity of cells is passed (block 1401) from a cell reservoir (FIG. 5, 534) to at least one marking chamber (FIG. 1, 104), where it is determined (block 1402) whether a cell should be marked, and marker (FIG. 3, 310) is applied (block 1403) to selected cells. These operations may be performed as described above in connection with FIG. 2.

Once marked, the marked cells (FIG. 3, 312) are detected (block 1404). That is, as described above, the marker (FIG. 3, 310) may alter an optical and/or electrical property of a particular cell and the detector (FIG. 4, 418) is selected which is capable of detecting this alteration. The marked cells (FIG. 3, 312) are then sorted (block 1405) based on their marker response. That is, different cells may be differentiated based on their response to the marker (FIG. 3, 310) that is applied. The different cells may be processed differently downstream. Accordingly, by sorting (block 1405) the marked cells (FIG. 3, 312) such differential processing is facilitated. The sorting (block 1405) may be implemented by activating pumps (FIG. 5, 545) in branched channels (FIG. 5, 536) designated to receive particular cells. For example, a first pump (FIG. 5, 545) may be activated to draw a first marked cell (FIG. 3, 312) through a first branched channel (FIG. 5, 536). The first pump (FIG. 5, 545) is activated when the first marked cell (FIG. 3, 312) is detected by the detector (FIG. 4, 418). Similarly, a second pump (FIG. 5, 545) may be activated to draw a second marked cell (FIG. 3, 312) through a second branched channel (FIG. 5, 536). The second pump (FIG. 5, 545) is activated when the second marked cell (FIG. 3, 312) is detected by the detector (FIG. 4, 418).

In addition to the pumps (FIG. 5, 545) in the branched channels (FIG. 5, 536), other downstream components may be activated (block 1406) based on the detection of marked cells (FIG. 3, 312). For example, particular lysing elements (FIG. 4, 424) may be activated when it is determined that a marked cell (FIG. 3, 312) intended to be lysed by that particular lysing element (FIG. 4, 424) is detected. In this example, the lysing element (FIG. 4, 424) may be selected with certain agitation parameters to particularly target that particular marked cell.

The marker (FIG. 3, 310) may also be used to track (block 1407) the marked cell (FIG. 3, 312) throughout the cell analysis system (FIG. 4, 414). That is, the marker (FIG. 3, 310) provides a way to follow the progression of a particular cell throughout its path along the cell analysis device (FIG. 4, 416), whether that includes ejection onto a different analytic component, or on the same substrate but in a different analysis device.

In summary, using such a cell analytic system 1) allows single cell analysis of a sample; 2) allows combined cell analysis, i.e., a genetic analysis and a mechanical property analysis; 3) can be integrated onto a lab-on-a-chip; 4) is scalable and can be parallelized for high throughput, 5) is low cost and effective; 6) reduces stain consumption; 7) allows tracking of a cell through a cell analysis system; 8) is robust against the rapidly changing profile of some cells; 9) accommodates different stains; 10) provides for real-time sample preparation; and 11) automates the cell preparation operation. However, the devices disclosed herein may address other matters and deficiencies in a number of technical areas.

What is claimed is:

1. A cell marking system, comprising:
a microfluidic channel to serially feed individual cells from a volume of cells into at least one marking chamber;
the at least one marking chamber to hold an individual cell to be marked; and
a marker application device per marking chamber to selectively apply a marker to the individual cell disposed within a respective marking chamber.

2. The cell marking system of claim 1, wherein:
the marker application device is on a second substrate distinct from a first substrate on which the respective marking chamber is formed;
the marker application device comprises a thermal inkjet ejector to eject the marker into the respective marking chamber; and
the marking chamber comprises an orifice through which the marker is received into the marking chamber.

3. The cell marking system of claim 1, wherein:
the cell marking system further comprises a marker reservoir to hold a volume of marker; and
the marker application device comprises a pump disposed in a marker channel formed on a same substrate on which the respective marking chamber is formed.

4. The cell marking system of claim 1:
further comprising a detector downstream of the at least one marking chamber to detect which cells have been marked; and
wherein an output of the detector selectively activates a particular feedback-controlled lysing element.

5. The cell marking system of claim 4, wherein based on a marker response of a marked cell, the detector is to perform at least one of:
triggering activation of a particular pump to draw a marked cell into a particular branched channel of a cellular analytic system; and
activating a waste ejector to eject unmarked cells.

6. The cell marking system of claim 1, wherein:
the at least one marking chamber comprises multiple marking chambers; and
the marker application devices eject different markers.

7. The cell marking system of claim 1, further comprising an integrated pump disposed in the microfluidic channel to move cells through the cell marking system.

8. The cell marking system of claim 1, further comprising a cell presence sensor to trigger activation of the marker application devices.

9. A method, comprising:
passing, in serial fashion, a quantity of cells from a cell reservoir to at least one cell marking system of a microfluidic cell analysis system; and
for each cell marking system:
determining whether a cell is to be marked; and
applying a marker to selected cells, wherein the marker remains on a cell wall and changes at least one of an optical and electrical property of a selected cell.

10. The method of claim 9, further comprising:
detecting marked cells; and
activating a downstream component of the microfluidic cell analysis system based on detection of marked cells.

11. The method of claim 9 further comprising:
sorting marked cells based on a marker response of each marked cell; and
tracking marked cells through the microfluidic cell analysis system.

12. A cell analysis system, comprising:
at least one cell analysis device, each cell analysis device comprising:
a microfluidic channel to serially feed individual cells from a volume of cells into at least one marking chamber;
at least one marking chamber to hold an individual cell to be marked;
a marker application device per marking chamber to apply a marker to the individual cell disposed within a respective marking chamber;
a detector to detect which cells have been marked;
a feedback-controlled lysing device comprising:
a lysing chamber;
at least one lysing element in the lysing chamber to agitate the individual cell; and
a sensor to determine a state within the lysing chamber;
a controller to analyze the individual cell, the controller comprising:
a lysate analyzer to analyze properties of a lysate of the individual cell;
a rupture analyzer to analyze parameters of an agitation when a cell membrane ruptures; and
a component controller to activate components of the cell analysis system based on an output of the detector.

13. The cell analysis system of claim 12, further comprising a number of branched channels, wherein each cell is directed to a particular branched chamber based on a marker response associated with that cell.

14. The cell analysis system of claim 12, further comprising a waste ejector per branched channel to eject unmarked cells from the particular branched channel.

15. The cell analysis system of claim 12, further comprising a waste reservoir.
